(19) United States
(12) Patent Application Publication
(10) Pub. No.: US 2018/0110774 A1
(43) Pub. Date: Apr. 26, 2018

(54) COMPOSITIONS AND METHODS FOR TREATMENT OF PULMONARY HYPERTENSION

(71) Applicant: STC.UNM, Albuquerque, NM (US)

(72) Inventors: Laura V. Gonzalez Bosc, Albuquerque, NM (US); Levi D. Maston, Albuquerque, NM (US); Thomas Resta, Albuquerque, NM (US)

(73) Assignee: STC. UNM, Albuquerque, NM (US)

(21) Appl. No.: 15/570,042

(22) PCT Filed: Apr. 28, 2016
(86) PCT No.: PCT/US16/29682

§ 371 Date: Oct. 27, 2017

Related U.S. Application Data
(60) Provisional application No. 62/153,826, filed on Apr. 28, 2015.

(51) Int. Cl. A61K 31/495; A61P 9/12; A61K 45/06; A61K 9/00

(52) U.S. Cl. CPC A61K 31/495; A61K 9/0078; A61K 45/06; A61P 9/12

(57) ABSTRACT

Methods and composition for treating or preventing pulmonary hypertension are provided. In certain aspects, compounds that inhibit T_{H}17 cell maturation or activity, such as retinoic acid receptor-related orphan nuclear receptor (ROR) inhibitors, are used to for the treatment of pulmonary hypertension.

This application claims the benefit of U.S. Provisional Patent Application No. 62/153,826, filed Apr. 28, 2015, the entirety of which is incorporated herein by reference.

The invention was made with government support under Grant No. 1F30HL123109-01 awarded by the National Institutes of Health. The government has certain rights in the invention.

### BACKGROUND OF THE INVENTION

### 1. Field of the Invention

The present invention relates generally to the field of molecular biology, immunology and medicine. More particularly, it concerns methods for treatment of lung disorders, such as pulmonary hypertension.

### 2. Description of Related Art

Pulmonary hypertension (PH) is a condition in which elevated pressure is found in the pulmonary artery. PH is defined as a resting mean pulmonary artery pressure greater than 25 mmHg. It can lead to right ventricular hypertrophy and right-sided heart failure if it is not successfully treated. Pulmonary hypertension may arise in relation to a variety of conditions. The World Health Organization recognizes five classes of PH (Bolignano et al., 2013): (I) Idiopathic, familial, and associated pulmonary arterial hypertension or PAH; (II) PH associated with left-sided heart disease; (III) PH associated with lung diseases, such as COPD and/or hypoxia (e.g., from sleep apnea); (IV) Chronic thromboembolic PH arising from obstruction of pulmonary arterial vessels; and (V) PH with unclear or multifactorial causes (e.g., dialysis-dependent chronic kidney disease). Hypoxic pulmonary hypertension, for instance, is caused by a variety of chronic lower respiratory diseases including chronic obstructive pulmonary disease (COPD), as well as by chronic exposure to high-altitude (see, Hopkins et al., 2002 and Poor et al., 2012) and acute lung injury (ALI). In fact, chronic lower respiratory diseases (CLRD) are the third leading cause of deaths in the United States (Federal Centers for Disease Control and Prevention).

Despite the large number of patients affected by PH most therapeutics that have been used in treatment, such as vasodilators, merely address the symptoms of PH. Thus, there remains a need for additional therapeutics for use in treating PH and, in particular, for therapies that that can target the underlying mechanism of the disease.

### SUMMARY OF THE INVENTION

In a first embodiment there is provided a method for treating or preventing pulmonary hypertension in a subject comprising administering an effective amount of a compound that inhibits T_{H}17 cell development or activity. For example, in some aspects, the compound is a retinoic acid receptor-related orphan nuclear receptor (ROR) inhibitor. In still further aspects, the compound is a RORα or RORγ (e.g., RORγτ) inhibitor. For example, the RORα or RORγτ inhibitor can be an inhibitory polynucleotide that reduces expression of RORα and/or RORγ (e.g., RORγτ) expression. Thus, in some aspects, the ROR inhibitor is a polynucleotide, such as an antisense RNA, a siRNA or a shRNA, that comprises a sequence complimentary to all of part of a RORα-cosing mRNA (see, e.g., NCBI accession nos. NM_134261.2, NM_134260.2, NM_002943.3, and NM_134262.2, each incorporated herein by reference) and/or RORγ-coding mRNA (see, e.g., NCBI accession nos. (NM_005060.3 and NM_001001523.1 (RORγτ), each incorporated herein by reference). In further aspects, the compound can be a compound that selectively inhibits RORα or selectively inhibits RORγ (e.g., RORγτ).

In some aspects, an ROR inhibitor for use according to the embodiments (e.g., a RORγ or RORγτ inhibitor) comprises a compound having the formula (I):

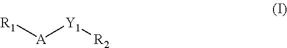

wherein:
A is —CH₂—, —S(O)₂NR₃— or —C(O)NR₃—;
   R₃ is hydrogen, alkyl_{(C≤12)}, substituted alkyl_{(C≤12)}, acyl_{(C≤12)}, or substituted acyl_{(C≤12)};
Y₁ is arenediyl_{(C≤18)}, heteroarenediyl_{(C≤18)}, or a substituted version thereof;
R₁ is aryl_{(C≤12)}, heteroaryl_{(C≤12)}, heterocycloalkyl_{(C≤12)}, or a substituted version of either of these groups; or an aryl_{(C≤12)}, heteroaryl_{(C≤12)}, heterocycloalkyl_{(C≤12)}, or a substituted version of either of these groups wherein the group is further substituted with an acyl_{(C≤8)}, amido_{(C≤8)}, alkylsulfonyl_{(C≤8)}, arylsulfonyl_{(C≤8)}, heteroarylsulfonyl_{(C≤8)}, alkylsulfonylamino_{(C≤8)}, arylsulfonylamino_{(C≤8)}, heteroarylsulfonylamino_{(C≤8)}, or a substituted version of any of these groups;
R₂ is

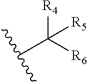

wherein:
   R₄ and R₆ is a haloalkyl_{(C≤6)}; and
   R₅ is hydroxy, alkoxy_{(C≤6)}, substituted alkoxy_{(C≤6)}, acyloxy_{(C≤6)}, or substituted acyloxy_{(C≤6)};
or a pharmaceutically acceptable salt thereof.
   In some embodiments, R₁ is heteroaryl_{(C≤12)} or a substituted heteroaryl_{(C≤12)} such as when R₁ is:

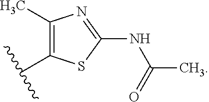 [SMILES: *c1sc(NC(C)=O)nc1C]

In other embodiments, R₁ is heterocycloalkyl_{(C≤12)} or substituted heterocycloalkyl_{(C≤12)} such as when R₁ is:

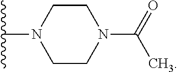 [SMILES: *N1CCN(C(C)=O)CC1]

In some embodiments, A is —S(O)₂NR₃—. In other embodiments, A is —CH₂—. In some embodiments, R₃ is hydrogen. In some embodiments, Y₁ is arenediyl_{(C≤18)}such as when Y₁ is:

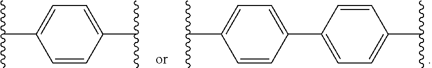 [SMILES: *c1ccc(*)cc1 ; *c1ccc(-c2ccc(*)cc2)cc1]

In some embodiments, R₄ is —CF₃. In some embodiments, R₆ is —CF₃. In some embodiments, R₅ is hydroxy. In some embodiments, R₂ is:

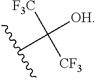 [SMILES: *C(O)(C(F)(F)F)C(F)(F)F]

In some embodiments, Y₁ and R₂ are taken together and are:

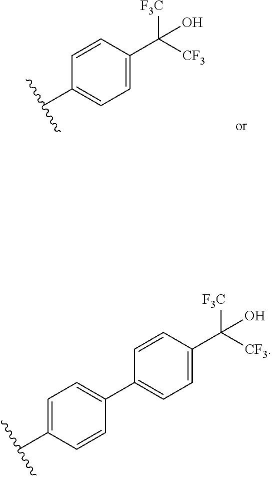 [SMILES: *c1ccc(C(O)(C(F)(F)F)C(F)(F)F)cc1 ; *c1ccc(-c2ccc(C(O)(C(F)(F)F)C(F)(F)F)cc2)cc1]

In some embodiments, the compound is further defined as:

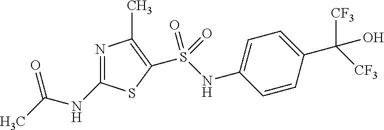 [SMILES: CC(=O)Nc1nc(C)c(S(=O)(=O)Nc2ccc(C(O)(C(F)(F)F)C(F)(F)F)cc2)s1]

or a pharmaceutically acceptable salt thereof. In other embodiments, the compound is further defined as:

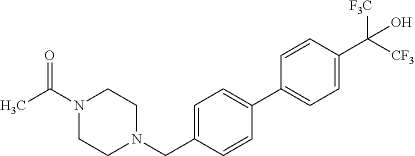 [SMILES: CC(=O)N1CCN(Cc2ccc(-c3ccc(C(O)(C(F)(F)F)C(F)(F)F)cc3)cc2)CC1]

or a pharmaceutically acceptable salt thereof. In some specific aspects, a compound for use according to the embodiments is SR 1001 (N-(5-(N-(4-(1,1,1,3,3,3-hexafluoro-2-hydroxypropan-2-yl)phenyl)sulfamoyl)-4-methylthiazol-2-yl)acetamide) or SR 1555.

Certain aspects of the embodiments concern administering a ROR inhibitor to a subject for treatment or prevention of pulmonary hypertension. In preferred aspects, the subject for treatment is a mammalian subject, such as a human. In some aspects, the subject has or has been diagnosed with pulmonary hypertension (PH) or pulmonary arterial hypertension (PAH). In still further aspects, the subject has a disease or injury that puts the subject at risk for the development of PH or PAH. For example, the subject may have, or has previously had, a lung infection or chronic lung infections. In further aspects, the subject has, or has previously been, chronically exposed to high altitude (e.g., a subject having spent a month or more over an altitude of 1,000 or 2,000 meters). In yet further aspects, the subject has, or has been previous been diagnosed with, chronic obstructive pulmonary disease (COPD), acute respiratory distress syndrome (ARDS), acute lung injury (ALI) (e.g., a chemical induced acute lung injury or inhalational smoke induced acute lung injury) or chronic lower respiratory diseases (CLRD).

In further aspects, a method of the embodiments may comprise administering at least a second therapeutic to a subject, such as a subject who has PH or is risk for developing PH. In some cases, a second therapeutic can be administered before, after or essentially simultaneously with a compound of the embodiments. In certain aspects, the second therapeutic can be co-formulated with a compound of the embodiments. A second therapeutic for use according to the embodiments can be, for example, a vasodilator, a prostanoid, an endothelin receptor antagonist, a phosphodiesterase-5 inhibitors or a sGC stimulator. In certain specific aspects, the second therapeutic comprises bosentan, macitentan, prostacyclin, sildenafil, tadalafil, treprostinil, iloprost and/or riociguat.

Pharmaceutically acceptable formulations of the embodiments may include, without limitation, salts, buffers, preservatives, thickener, stabilizers and surfactants. In certain aspects, the formulations are aqueous formulations. In some cases, the formulations are lyophilized and may, in some cases, be solubilized in a solution prior to administration. In certain aspects, pharmaceutical formulation of the embodiments is essentially free of a cationic, anionic, zwitterionic or non-ionic surfactants surfactant. In preferred aspects, pharmaceutical formulations of the embodiments is filtered and/or sterilized. In specific aspects, a pharmaceutical formulation comprises a sterile saline or phosphate buffered saline (PBS) solution. In other aspects, the solution is essentially free of a pH buffering agent. In further aspects, a pharmaceutical formulation comprises a stabilizer. For example, the stabilizer can comprise, amino acids, such as glycine and lysine, carbohydrates or a lyoprotectant (e.g., dextrose, mannose, galactose, fructose, lactose, sucrose, maltose, sorbitol, or mannitol). In specific aspects, the pharmaceutical formulation consists essentially of a sterile saline solution and a compound of the embodiments that inhibits T_{H}17 cell activity or maturation. Further components for inclusion in pharmaceutical formulations of the embodiments are detailed herein below.

In some embodiments, a compound of the embodiments, such as a ROR inhibitor, is administered locally (e.g., by aerosol administration to the lungs). In some embodiments, the compound is administered systemically. In certain embodiments, the compound is administered orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, orally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, via localized perfusion, bathing target cells directly, or any combination thereof. For example, in some variations, the compound is administered intravenously, intra-arterially or orally. For example, in some variations, the compound is administered orally.

In further embodiments, a compound of the embodiments is formulated as a hard or soft capsule, a tablet, a syrup, a suspension, a solid dispersion, a wafer, or an elixir. In some variations, the soft capsule is a gelatin capsule. In variations, the compound is formulated as a solid dispersion. In some variations the hard capsule, soft capsule, tablet or wafer further comprises a protective coating. In some variations, the formulated compound comprises an agent that delays absorption. In some variations, the formulated compound further comprises an agent that enhances solubility or dispersibility. In some variations, the compound is dispersed in a liposome, an oil-in-water emulsion or a water-in-oil emulsion.

Thus, in some, embodiments a nebulized or aerosolized composition is provided comprising a compound of the embodiments that inhibits T_{H}17 cell activity or maturation (e.g., a ROR inhibitor). In some aspects, the nebulized solution is produced using a vibrating mesh nebulizer. The vibrating mesh nebulizer may be an active or a passive vibrating mesh nebulizer. In some aspects, the vibrating mesh nebulizer may be an Aeroneb® Professional Nebulizer System or an EZ Breathe Atomizer. In further aspects, the nebulized solution is produced using a jet nebulizer or an ultrasonic nebulizer. In some aspects, a nebulized solution of the embodiments may have a median particle size of between about 2.5 μm and 100 μm, 2.5. μm and 50 μm, 2.5 μm and 20 μm, 2.5 μm and 8 μm, or 3.0 μm and 6 μm. In certain aspects the nebulized solution comprises an emulsion. In still further aspects, the solution comprises a surfactant, such as a polysorbate surfactant (e.g., TWEEN 20 or TWEEN 80). In yet further aspects, a ROR inhibitor of the embodiments is a provided to the airway as an aerosol of a lyophilized powder.

As used herein an “effective amount” of a compound, such as a ROR inhibitor, refers to an amount that is effective, when administered to a subject, to reduce T_{H}17 cell levels, reduce T_{H}17 cell activity or reduce pulmonary artery pressure (e.g., resting mean pulmonary artery pressure) in the subject.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Perivascular infiltration of T cells (CD3⁺) is increased by Chronic hypoxia (CH). Arrows indicate CD3 positive cell. *p<0.05. n=4.

FIG. 2—CD4⁺ T cells contribute to CH-induced PH. RAG1 KO mice, which lack mature T and B cells, are protected from CH-induced increases in RVSP and RV/LV+S weight. CD4⁺ T cells but not CD8+ T cells contribute to CH-induced PH. *p<0.05 vs. normoxia, #p<0.05 vs. WT, & p<0.05 vs. No AT. n=6-8.

FIG. 3A-B—Flow cytometric detection of T_{H}17 cells in the lungs of normoxia and CH-WT mice. A) Cell plots showing CD4⁺ vs. IL-17⁺ axes. B) Quantification of flow data. *p<0.05. n=7-8 mice.

FIG. 4—T_{H}17 cells cause PH. RAG1 KO mice received saline or purified T_{H}17 cells. *p<0.05 vs. normoxia, #p<0.05 vs. saline. n=4 mice.

FIGS. 5A-D—Inhibition of T_{H}17 cell development decreases CH-induced increases in perivascular T cells. Following treatment, lungs sections were stained with an anti-CD3 antibody to allow quantification of CD3+ T cells in the perivascular region of the lungs. The RORyt specific inhibitor, SR1001 was delivered daily by sub-cutaneous injection (0.625 mg/day for 25 g mouse) for the length of normoxic or CH exposure. (A) Perivascular CD3+ cells from mice exposed to 2 days, (B) 5 days or (C) 21 days of normoxia or CH. (D) Representative images of lung sections from mice exposed to CH with or without SR1001. Values are means±SEM, *p<0.05 vs. normoxia vehicle. n=3-4/group, 5-15 arteries<150 μm outer circumference, per mouse, analyzed by 2-way ANOVA followed by multiple comparisons Student-Newman-Keuls test.

FIGS. 6A-D—Inhibition of T_{H}17 cell polarization attenuates the development of CH-induced PH. SR1001 prepared in propylene glycol, 0.625 mg/day for 25 g mouse, was delivered sub-cutaneously via an implantable osmotic pump. Mice were exposed to normoxia or chronic hypoxia for 21 days, at which point (A) RVSP, (B) Fulton's index, (C) pulmonary arterial % wall thickness (<150 μm diameter) and (D) hematocrit were measured. Values are means±SEM; *p<0.05 vs. normoxia vehicle #p<0.05 vs. normoxic SR1001, n=4-5/group, analyzed by 2-way ANOVA followed by multiple comparisons Student-Newman-Keuls test.

FIGS. 7A-D—T_{H}17 cell inhibition attenuates increases in RVSP in established PH. Wild-type mice were exposed to CH for 21 days followed by administration of SR1001 (25 mg/kg/day s.c.) for an additional 14 days in CH. (A) RVSP. (B) Fulton's index. (C) Pulmonary arterial remodeling. (D) % Hematocrit. Values are means±SEM; *p<0.05 vs. vehicle, n=5-6/group, analyzed by unpaired T-test.

### DESCRIPTION OF ILLUSTRATIVE EMBODIMENTS

### I. The Present Embodiments

Pulmonary hypertension is a disease associated with significant morbidity and mortality. However, to date available therapies were only able to treat disease symptoms. For example, vasodilators and have been employed to reduced pulmonary artery pressure. In contrast studies presented herein demonstrate a role of inflammatory T-cells, in particular, T_{H}17 cells in mediating PH. The inventors have determined that drugs used to prevent T_{H}17 maturation or inhibit T_{H}17 activity may have significant utility in the treatment of PH. For example, retinoic acid receptor-related orphan nuclear receptor (ROR) inhibitors can be employed to inhibit T_{H}17 maturation and activity. Importantly, since the compounds and methods of the present embodiments treat the underlying disease mechanism, they offer the promise of more targeted and effective therapy as well as use in preventing the onset of PH in at risk populations (such as patient with chromic respiratory infections).

### II. Pulmonary Hypertension

Pulmonary hypertension is a life-threatening disease characterized by a marked and sustained elevation of pulmonary artery pressure and an increase in pulmonary vascular resistance leading to right ventricular (RV) failure and death. Current therapeutic approaches for the treatment of chronic pulmonary arterial hypertension mainly provide symptomatic relief, as well as some improvement of prognosis. PH is caused by a constellation of diseases that affect the pulmonary vasculature. PH can be caused by or associated with collagen vascular disorders, such as systemic sclerosis (scleroderma), uncorrected congenital heart disease, liver disease, portal hypertension, HIV infection, Hepatitis C, certain toxins, splenectomy, hereditary hemorrhagic telangiectasia, and primary genetic abnormalities. For instance, a mutation in the bone morphogenetic protein type 2 receptor (a TGF-b receptor) has been identified as a cause of certain familial primary pulmonary hypertension (PPH) (Deng et al., 2000). It is estimated that 6% of cases of PPH are familial, and that the rest are “sporadic.” The incidence of PPH is estimated to be approximately 1 case per 1 million population. Secondary causes of PAH have a much higher incidence. The pathologic signature of PAH is the plexiform lesion of the lung, which consists of obliterative endothelial cell proliferation and vascular smooth muscle cell hypertrophy in small precapillary pulmonary arterioles. PH is a progressive disease associated with a high mortality and patients with PH may develop right ventricular (RV) failure.

The evaluation and diagnosis of PH is reviewed by McLaughlin and Rich (2004) and McGoon et al. (2004). A clinical history, such as symptoms of shortness of breath, a family history of PH, presence of risk factors, and findings on physical examination, chest X-ray and electrocardiogram may lead to the suspicion of PH. The next step in the evaluation will usually include an echocardiogram. The echocardiogram can be used to estimate the pulmonary artery pressure from the Doppler analysis of the tricuspid regurgitation jet. The echocardiogram can also be used to evaluate the function of the right and left ventricle, and the presence of valvular heart disease, such as mitral stenosis and aortic stenosis. The echocardiogram can also be useful in diagnosing congenital heart disease, such as an uncorrected atrial septal defect or patent ductus arteriosus. Findings on echocardiogram consistent with a diagnosis of PAH would include: 1) Doppler evidence for elevated pulmonary artery pressure; 2) right atrial enlargement; 3) right ventricular enlargement and/or hypertrophy; 4) absence of mitral stenosis, pulmonic stenosis, and aortic stenosis; 5) normal size or small left ventricle; 6) relative preservation of or normal left ventricular function. To confirm the diagnosis of PH a cardiac catheterization to directly measure the pressures in the right side of the heart and in the pulmonary vasculature is typically performed. An accurate measurement of the pulmonary capillary wedge pressure (PCWP), which gives an accurate estimate of the left atrial and left ventricular end-diastolic pressure, is also required. If an accurate PCWP cannot be obtained, then direct measurement of LV end-diastolic pressure by left heart catheterization is advised. By definition, patients with PAH should have a low or normal PCWP. However, in the late stages of PH, the PCWP can become somewhat elevated though usually not greater than 16 mm Hg (McLaughlin and Rich, 2004; McGoon et al., 2004). The upper limit of normal for mean pulmonary artery pressure in an adult human is 19 mm Hg. A commonly used definition of mean pulmonary artery pressure is one-third the value of the systolic pulmonary artery pressure plus two-thirds of the diastolic pulmonary artery pressure. Severe PAH may be defined as a mean pulmonary artery pressure greater than or equal to 25 mm Hg with a PCWP less than or equal to 15-16 mm Hg, and a pulmonary vascular resistance (PVR) greater than or equal to 240 dynes sec/cm⁵. Pulmonary vascular resistance is defined as the mean pulmonary artery pressure minus the PCWP divided by the cardiac output. This ratio is multiplied by 80 to express the result in dyne sec/cm⁵. The PVR may also be expressed in millimeters Hg per liter per minute, which is referred to as Wood Units. The PVR in a normal adult is 67±23 dyne sec/cm⁵ or 1 Wood Unit (McLaughlin and Rich, 2004; McGoon et al., 2004).

In some cases, the status of pulmonary arterial hypertension can be assessed in patients according to the World Health Organization (WHO) classification (modified after the New York Association Functional Classification) as detailed below: Class I-Patients with pulmonary hypertension but without resulting limitation of physical activity. Ordinary physical activity does not cause undue dyspnea or fatigue, chest pain or near syncope.

Class II—Patients with pulmonary hypertension resulting in slight limitation of physical activity. They are comfortable at rest. Ordinary physical activity causes undue dispend or fatigue, chest pain or near syncope.

Class III—Patients with pulmonary hypertension resulting in marked limitation of physical activity. They are comfortable at rest. Less than ordinary activity causes undue dyspnea or fatigue, chest pain or near syncope.

Class IV—Patients with pulmonary hypertension with inability to carry out any physical activity without symptoms. These patients manifest signs of right heart failure. Dyspnea and/or fatigue may even be present at rest. Discomfort is increased by any physical activity.

At one time, the only effective long-term therapy for PAH in conjunction with anticoagulant therapy was continuous intravenous administration of prostacyclin, also known as epoprostenol (PGI₂) (Barst et al., 1996; McLaughlin et al., 1998). Later, the non-selective endothelin receptor antagonist, bosentan, showed efficacy for the treatment of PAH (Rubin et al., 2002). As the first orally bioavailable agent with efficacy in the treatment of PAH, bosentan represented a significant advance. However, the current leading therapeutic category for PAH is treatment with a selective endothelin type A receptor antagonist (Galie et al., 2005; Langleben et al., 2004). Inhibitors of phosphodiesterase type V (PDE-V), including sildenafil and tadalafil, have been approved for the treatment of PAH (Lee et al., 2005; Kataoka et al., 2005). PDE-V inhibition results in an increase in cyclic GMP, which leads to vasodilation of the pulmonary vasculature. Treprostinil, an analogue of PGI₂, can be administered subcutaneously to appropriately selected patients with PAH (Oudiz et al., 2004; Vachiery and Naeije, 2004). In addition, Iloprost, another prostacyclin analogue, can be administered in nebulized form by direct inhalation (Galie et al., 2002). Riociguat, a stimulator of soluble guanylate cyclas (sGC), is also approved for the treatment of PAH. These agents are used to treat PAH of multiple etiologies, including PH associated with or caused by familial PAH (primary pulmonary arterial hypertension or PPH), idiopathic PAH, scleroderma, mixed connective tissue disease, systemic lupus erythematosus, HIV infection, toxins, such as phentermine/fenfluramine, congenital heart disease, Hepatitis C, liver cirrhosis, chronic thrombo-embolic pulmonary artery hypertension (distal or inoperable), hereditary hemorrhagic telangiectasia, and splenectomy. All approved agents for PAH are essentially vasodilatory in effect. Consequently, they only address a portion of the overall pathology of PH. Without being bound by theory, compounds and methods detailed herein, have a direct effect on immune cells (T_{H}17 cells) that mediate disease and thus, potentially, offer a way of addressing PH pathology in a more comprehensive fashion.

### III. Compounds of the Embodiments

Certain aspects of the embodiments concern compounds that have ROR inhibitor activity. For example, in certain specific aspects, the compound is a SR 1001, SR 1555 or a derivative thereof (Solt et al., 2011 and Solt et al., 2012, each incorporated herein by reference). Other contemplated compounds which may be used to inhibit the activity of ROR include those described in PCT Patent Application No. WO 2011/115892 which is incorporated herein by reference.

The compounds used herein are shown, for example, above in the summary of the invention section and in the claims below. They may be made using the methods known to those of skill in the art. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

Compounds of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It is appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### A. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxylsulfonyl” means —SO₂OH; “aminosulfonyl” means —SO₂NH₂ and “sulfinyl” means —S(O)—.

In the context of chemical formulas, the symbol “-” means a single bond, “═” means a double bond, and “≡” means triple bond. The symbol “----” represents n optional bond, which if present is either single or double. The symbol “ [symbol] ” represents a single bond or a double bond. Thus, for example, the formula

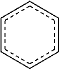

includes

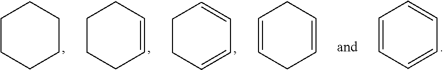

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

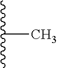

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

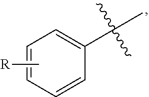

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

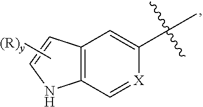

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and compound classes below, the number of carbon atoms in the group is as indicated as follows: “Cn” defines the exact number (n) of carbon atoms in the group/class. “C≤n” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. Also compare “phosphine_{(C≤10)}”, which designates phosphine groups having from 0 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. Typically the carbon number indicator follows the group it modifies, is enclosed with parentheses, and is written entirely in subscript; however, the indicator may also precede the group, or be written without parentheses, without signifying any change in meaning. Thus, the terms “C5 olefin”, “C5-olefin”, “olefin_{(C5)}”, and “olefin_{C5}” are all synonymous. When any group or compound class below is used with the term “substituted”, any carbon atoms of the chemical group replacing the hydrogen atom do not count towards the total carbon atom limit for that group or compound class.

The term “saturated” when used to modify a compound or an atom means the compound or atom has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

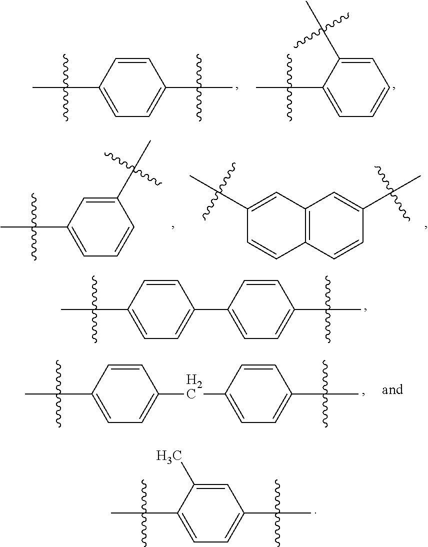

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

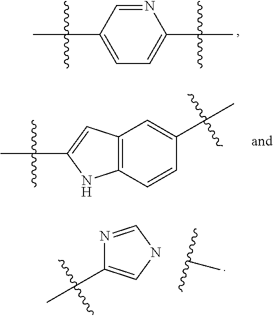

The term “N-heteroaryl” refers to a heteroaryl group with a nitrogen atom as the point of attachment. A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “aryloxy”, “heteroaryloxy”, “heterocycloalkoxy”, and “acyloxy”, when used without the “substituted” modifier, refers to groups, defined as —OR, in which R is aryl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “arylamino”, “heteroarylamino”, “heterocycloalkylamino”, “alkoxyamino”, “alkylsulfonylamino”, “arylsulfonylamino”, and “heteroarylsulfonylamino” when used without the “substituted” modifier, refers to groups, defined as —NHR, in which R is aryl, heteroaryl, heterocycloalkyl, alkoxy, alkylsulfonyl, arylsulfonyl, and heteroarylsulfonyl, respectively. Other groups are defined analogously. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms “alkylsulfonyl” and “alkylsulfinyl” when used without the “substituted” modifier refers to the groups —S(O)₂R and —S(O)R, respectively, in which R is an alkyl, as that term is defined above. The terms “arylsulfonyl”, “heteroarylsulfonyl”, and “heterocycloalkylsulfonyl” are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### IV. Pharmaceutical Formulations and Routes of Administration

Administration of the compounds of the present embodiments to a subject will follow general protocols for the administration of pharmaceuticals, taking into account the toxicity, if any, of the drug. It is expected that the treatment cycles would be repeated as necessary.

The compounds of the present embodiments may be administered by a variety of methods, e.g., orally, by inhalation (e.g., in an aerosol) or by injection (e.g. subcutaneous, intravenous, intraperitoneal, etc.). Depending on the route of administration, the active compounds may be coated by a material to protect the compound from the action of acids and other natural conditions which may inactivate the compound. They may also be administered by continuous perfusion/infusion of a disease site. It will be recognized by those skilled in the art that other methods of manufacture may be used to produce dispersions of the present embodiments with equivalent properties and utility (see, Repka et al., 2002 and references cited therein). Such alternative methods include but are not limited to solvent evaporation, extrusion, such as hot melt extrusion, and other techniques.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984). Dispersions may be prepared in, e.g., glycerol, liquid polyethylene glycols, mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. Formulations must be stable under the conditions of manufacture and storage and must be preserved against the contaminating action of microorganisms such as bacteria and fungi. The carrier may be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

In other aspects, the therapeutic compound may be formulated in a biocompatible matrix for use in a drug-eluting stent.

The actual dosage amount of a compound of the present embodiments or composition comprising a compound of the present embodiments administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the pharmaceutically effective amount is a daily dose from about 0.1 mg to about 500 mg of the compound. In some variations, the daily dose is from about 1 mg to about 300 mg of the compound. In some variations, the daily dose is from about 10 mg to about 200 mg of the compound. In some variations, the daily dose is about 25 mg of the compound. In other variations, the daily dose is about 75 mg of the compound. In still other variations, the daily dose is about 150 mg of the compound. In further variations, the daily dose is from about 0.1 mg to about 30 mg of the compound. In some variations, the daily dose is from about 0.5 mg to about 20 mg of the compound. In some variations, the daily dose is from about 1 mg to about 15 mg of the compound. In some variations, the daily dose is from about 1 mg to about 10 mg of the compound. In some variations, the daily dose is from about 1 mg to about 5 mg of the compound.

In some embodiments, the pharmaceutically effective amount is a daily dose is 0.01-25 mg of compound per kg of body weight. In some variations, the daily dose is 0.05-20 mg of compound per kg of body weight. In some variations, the daily dose is 0.1-10 mg of compound per kg of body weight. In some variations, the daily dose is 0.1-5 mg of compound per kg of body weight. In some variations, the daily dose is 0.1-2.5 mg of compound per kg of body weight.

In some embodiments, the pharmaceutically effective amount is a daily dose is of 0.1-1000 mg of compound per kg of body weight. In some variations, the daily dose is 0.15-20 mg of compound per kg of body weight. In some variations, the daily dose is 0.20-10 mg of compound per kg of body weight. In some variations, the daily dose is 0.40-3 mg of compound per kg of body weight. In some variations, the daily dose is 0.50-9 mg of compound per kg of body weight. In some variations, the daily dose is 0.60-8 mg of compound per kg of body weight. In some variations, the daily dose is 0.70-7 mg of compound per kg of body weight. In some variations, the daily dose is 0.80-6 mg of compound per kg of body weight. In some variations, the daily dose is 0.90-5 mg of compound per kg of body weight. In some variations, the daily dose is from about 1 mg to about 5 mg of compound per kg of body weight.

An effective amount typically will vary from about 0.001 mg/kg to about 1,000 mg/kg, from about 0.01 mg/kg to about 750 mg/kg, from about 0.1 mg/kg to about 500 mg/kg, from about 0.2 mg/kg to about 250 mg/kg, from about 0.3 mg/kg to about 150 mg/kg, from about 0.3 mg/kg to about 100 mg/kg, from about 0.4 mg/kg to about 75 mg/kg, from about 0.5 mg/kg to about 50 mg/kg, from about 0.6 mg/kg to about 30 mg/kg, from about 0.7 mg/kg to about 25 mg/kg, from about 0.8 mg/kg to about 15 mg/kg, from about 0.9 mg/kg to about 10 mg/kg, from about 1 mg/kg to about 5 mg/kg, from about 100 mg/kg to about 500 mg/kg, from about 1.0 mg/kg to about 250 mg/kg, or from about 10.0 mg/kg to about 150 mg/kg, in one or more dose administrations daily, for one or several days (depending, of course, of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day, less than 10 mg/kg/day, or less than 5 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 5 mg/kg/body weight, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present invention may comprise, for example, at least about 0.1% of a compound of the present invention. In other embodiments, the compound of the present invention may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The compound(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may taken orally and that the timing of which is or is not dependent upon food intake. Thus, for example, the agent can be taken every morning and/or every evening, regardless of when the subject has eaten or will eat.

### IV. Aerosol Dispersion and Nebulizing Devices

In certain aspects of the embodiments formulations (e.g., comprising an ROR inhibitor) can be aerosolized using any suitable device, including but not limited to a jet nebulizer, an ultrasonic nebulizer, a metered dose inhaler (MDI), and a device for aerosolization of liquids by forced passage through a jet or nozzle (e.g., AERX® drug delivery devices by Aradigm of Hayward, Calif.). For delivery of a formulation to a subject, as described further herein below, an pulmonary delivery device can also include a ventilator, optionally in combination with a mask, mouthpiece, mist inhalation apparatus, and/or a platform that guides users to inhale correctly and automatically deliver the drug at the right time in the breath. Representative aerosolization devices that can be used in accordance with the methods of the present invention include but are not limited to those described in U.S. Pat. Nos. 5,277,175; 5,284,133; 5,355,872; 5,660,166; 5,797,389; 5,823,179; 6,016,974; 6,041,776; 6,044,841; 6,241,159; 6,354,516; and 6,357,671 and U.S. Published Patent Application Nos. 20020020412 and 20020020409.

Using a jet nebulizer, compressed gas from a compressor or hospital air line is passed through a narrow constriction known as a jet. This creates an area of low pressure, and liquid medication from a reservoir is drawn up through a feed tube and fragmented into droplets by the air stream. Only the smallest drops leave the nebulizer directly, while the majority impact on baffles and walls and are returned to the reservoir. Consequently, the time required to perform jet nebulization varies according to the volume of the composition to be nebulized, among other factors, and such time can readily be adjusted by one of skill in the art.

A metered dose inhalator (MDI) can be used to deliver a composition of the invention in a more concentrated form than typically delivered using a nebulizer. For optimal effect, MDI delivery systems require proper administration technique, which includes coordinated actuation of aerosol delivery with inhalation, a slow inhalation of about 0.5-0.75 liters per second, a deep breath approaching inspiratory capacity inhalation, and at least 4 seconds of breath holding. Pulmonary delivery using a MDI is convenient and suitable when the treatment benefits from a relatively short treatment time and low cost. Optionally, a formulation can be heated to about 25° C. to about 90° C. during nebulization to promote effective droplet formation and subsequent delivery. See e.g., U.S. Pat. No. 5,299,566.

Aerosol compositions of the embodiments comprise droplets of the composition that are a suitable size for efficient delivery within the lung. In some cases, a surfactant formulation is delivered to lung bronchi, more preferably to bronchioles, still more preferably to alveolar ducts, and still more preferably to alveoli. Aerosol droplets are typically less than about 15 μm in diameter, less than about 10 μm in diameter, less than about 5 μm in diameter, or less than about 2 μm in diameter. For efficient delivery to alveolar bronchi of a human subject, an aerosol composition may preferably comprises droplets having a diameter of about 1 μm to about 5 μm.

Droplet size can be assessed using techniques known in the art, for example cascade, impaction, laser diffraction, and optical patternation. See McLean et al., 2000, Fults et al., 1991 and Vecellio et al., 2001, each incorporated herein by reference. Formulation stability following aerosolization can be assessed using known techniques in the art, including size exclusion chromatography; electrophoretic techniques; spectroscopic techniques such as UV spectroscopy and circular dichroism spectroscopy, and compound activity (measured in vitro or in vivo).

The term “vibrating mesh nebulizer” refers herein to any nebulizer that operates on the general principle of using a vibrating mesh or plate with multiple aperatures (an aperture plate) to generate a fine-particle, low-velocity aerosol. Some nebulizers may contain a mesh/membrane with between 1000 and 7000 holes, which mesh/membrane vibrates at the top of a liquid reservoir (see, e.g., U.S. Patent Publn. 20090134235 and Waldrep and Dhand 2008, each incorporated herein by reference). In some embodiments, the vibrating mesh nebulizer is an AERONEB® Professional Nebulizer, Omron MICROAIR®, Pari EFLOW® or an EZ Breathe Atomizer. In some aspects, a vibrating mesh nebulizer has a vibrating frequency of between about 50-250 kHz, 75-200 kHz 100-150 kHz or about 120 kHz. These devices have a high efficiency of delivering aerosol to the lung and the volume of liquid remaining in these devices is minimal, which is an advantage for expensive and potent compounds like plasminogen activators.

In certain aspects, a nebulized composition of the embodiments is produced using a vibrating mesh nebulizer. For example, the composition can be produced with an active vibrating mesh nebulizer (e.g., an Aeroneb® Professional Nebulizer System). Descriptions of such system and there operation can be found, for instance, in U.S. Pat. Nos. 6,921,020; 6,926,208; 6,968,840; 6,978,941; 7,040,549; 7,083,112; 7,104,463; and 7,360,536, each of which is incorporated herein by reference in its entirety. In yet further aspects, a composition of the embodiments can be produced with a passive vibrating mesh nebulizer, such as the Omron MicroAir® or the EZ Breathe Atomizer.

### V. Combination Therapy

In addition to being used as a monotherapy, the compounds of the present embodiments, such as ROR inhibitors, may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

Various combinations may be employed, such as when a compound of the present invention is “A” and “B” represents a secondary agent, non-limiting examples of which are described below:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

In some aspects, it is contemplated that anti-inflammatory agents may be used in conjunction with the treatments of the embodiments. For example, other COX inhibitors may be used, including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam. See also U.S. Pat. No. 6,025,395, which is incorporated herein by reference.

FDA approved treatments for pulmonary arterial hypertension include prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat). The use of any of these agents in conjunction with the treatments of the current embodiments is contemplated. When combined with a compound of the current embodiments, such as a ROR inhibitor, agents may be administered at the standard approved dose or in the standard approved range of doses, or may be administered at a lower than standard dose.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Preliminary Data

Methods: Age- and sex-matched mice C57BL/6J wild-type (WT), RAG1 KO mice (lack mature T and B cells), IL-17-GFP mice (express EGFP in cells expressing IL-17A), were used in these studies (The Jackson Laboratory). Mice were exposed to chronic hypoxia (CH) for 2 to 21 days in a hypobaric chamber maintained at a barometric pressure of 380 mmHg. Normoxic animals were housed in identical cages in the same facility under normobaric conditions (P_{B}=630 mmHg in Albuquerque, N. Mex.).

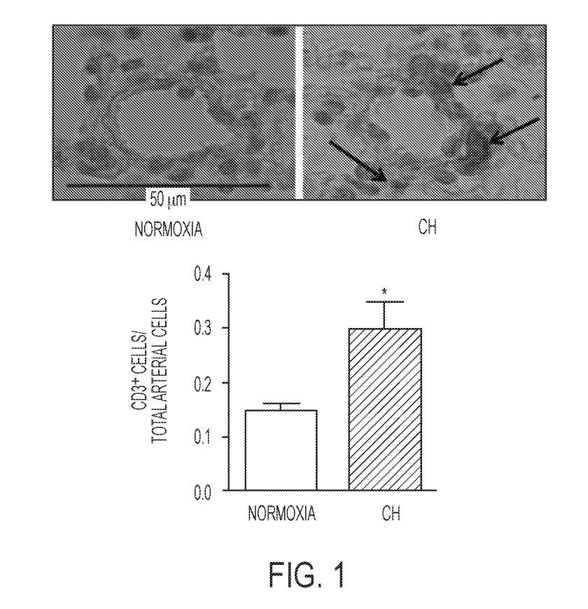

T cells were observed to increase in the perivascular area of pulmonary arteries of WT mice exposed to 5 days of CH (FIG. 1). T cells were quantified by immunohistochemistry detection of the pan T cell marker, CD3, in lung sections. These data show that T cells migrate to pulmonary arteries during CH-induced pulmonary hypertension.

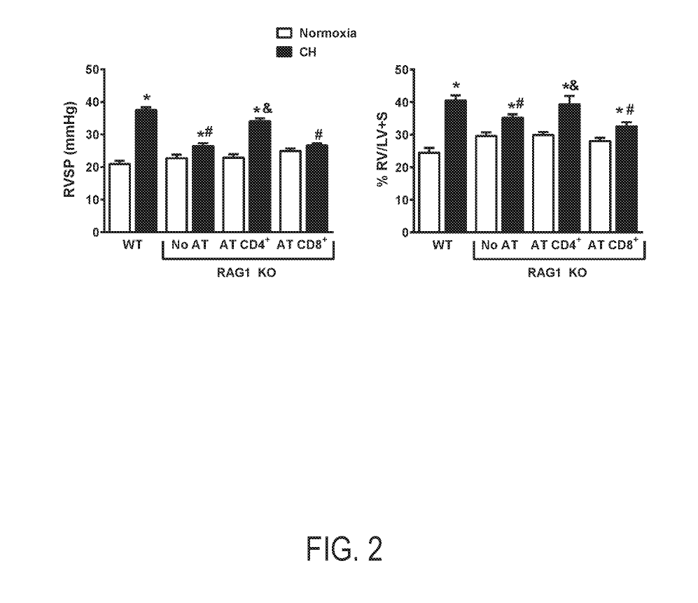

CD4+ T cells contribute to chronic hypoxia-induced pulmonary hypertension. To directly assess the contribution of T cells to CH-induced PH, recombination-activating gene 1 (RAG1) knockout mice, which lack the enzyme that plays a crucial role in the development of T and B cells were studied. Therefore, RAG1 KO mice lack mature T and B cells (Mombaerts et al., 1992). RAG1 KO mice were exposed to CH for 21 days or left in normoxia. FIG. 2A shows that WT mice exhibit a significant increase in RVSP following CH exposure, and that this increases is significantly attenuated in RAG1 KO mice that received no adoptive transfer (No AT). RVSP was measured by direct cardiac puncture under isoflurane anesthesia (Bierer et al., 2011). The inventors also isolated CD4⁺ or CD8+ cells from the lymph nodes of WT mice by negative selection using a magnetic bead cell isolation kit (Miltenyi Biotec). 2.5×10⁵ cells were injected into RAG1 KO mice. After 2 weeks mice were exposed to CH or normoxia. FIG. 2A shows that the adoptive transfer of CD4+ T cells but not CD8+ T cells is sufficient to restore the increased RVSP following CH. FIG. 2B shows similar results for Fulton's index. These results indicate that CD4⁺ T cells are a major contributor to CH-induced PH and are consistent with a report using the monocrotaline-induced PAH model (Cuttica et al., 2011).

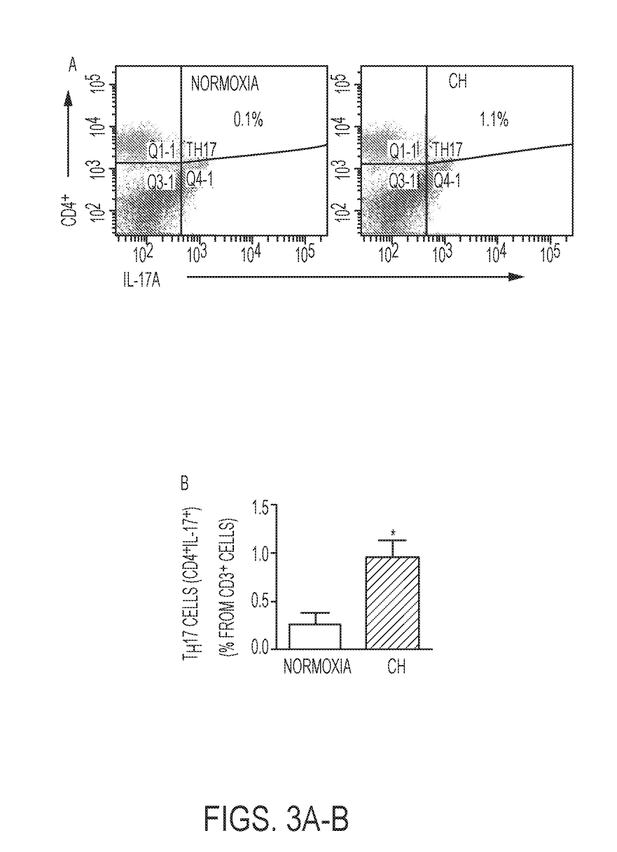

CH increases T_{H}17 cells in lungs. T_{H}17 cells are implicated in immune-mediated inflammatory diseases (Cheng et al., 2008; Eid et al., 2009; Lindén et al., 2006; Madhur et al., 2010; Shao et al., 2003; Tesmer et al., 2008) by attracting neutrophils, stimulating the release of matrix metalloproteinases, as well as increasing the release of factors from resident cells (Lindén et al., 2006). Interestingly, studies show increased number of circulating T_{H}17 cells in patients with PAH (Hautefort et al., 2014) and COPD (Vargas-Rojas et al., 2011), and in the lung of CH-exposed mice (Hashimoto-Kataoka et al., 2015). However, it is currently unknown the location of these cells within the lung and whether they are required for CH-induced PH. Lacking unique surface markers, the signature molecules for each of the CD4⁺ T_{H}-cell subsets are intracellular cytokines or transcription factors. Therefore, T_{H}17 cells were identified by detecting intracellular IL-17A and CD4 surface expression. T cells from digested lungs from normoxic and CH mice were incubated for 4 hr with phorbol 12-myristate 13-aceate (PMA; 50 ng/ml), ionomycin (1 μg/ml), and a protein-transport inhibitor, monensin (GolgiStop; BD Biosciences) before immunostaining to enhance the sensitivity of IL-17A detection. Therefore, the percent of CD4⁺ IL-17A⁺ cells was determined in whole lung digest of mice exposed to 5 days of CH or normoxia using flow cytometry. Nearly a 20-fold increase in T_{H}17 cells was found in digested lungs of mice exposed to CH compared to normoxic controls (FIG. 3A-B).

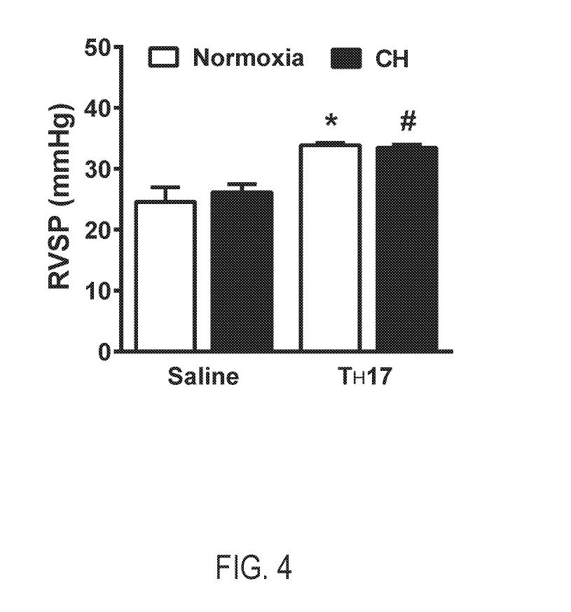

To determine the role of T_{H}17 cells in CH-induced PH, CD4⁺ T cells were purified from the lymph nodes of IL-17-EGFP mice by negative selection. CD4⁺ cells were polarized in culture to T_{H}17 cells (Veldhoen et al., 2009). Then, CD4⁺EGFP⁺ cells were purified by fluorescent-activated cell sorting (FACS) and 10⁴ T_{H}17 cells were injected (retro-orbital) into RAG1 KO mice. After 2 weeks, mice were exposed to CH for 21 days or left in normoxia. After 5 weeks, 90% of the splenocytes of these mice were CD3⁺CD4⁺EGFP⁺. T_{H}17-reconstituted mice show elevated RVSP after both normoxia and CH (FIG. 4), indicating that T_{H}17 cells are sufficient to cause PH.

### Example 2—Inhibition of T_{H}17 Cells

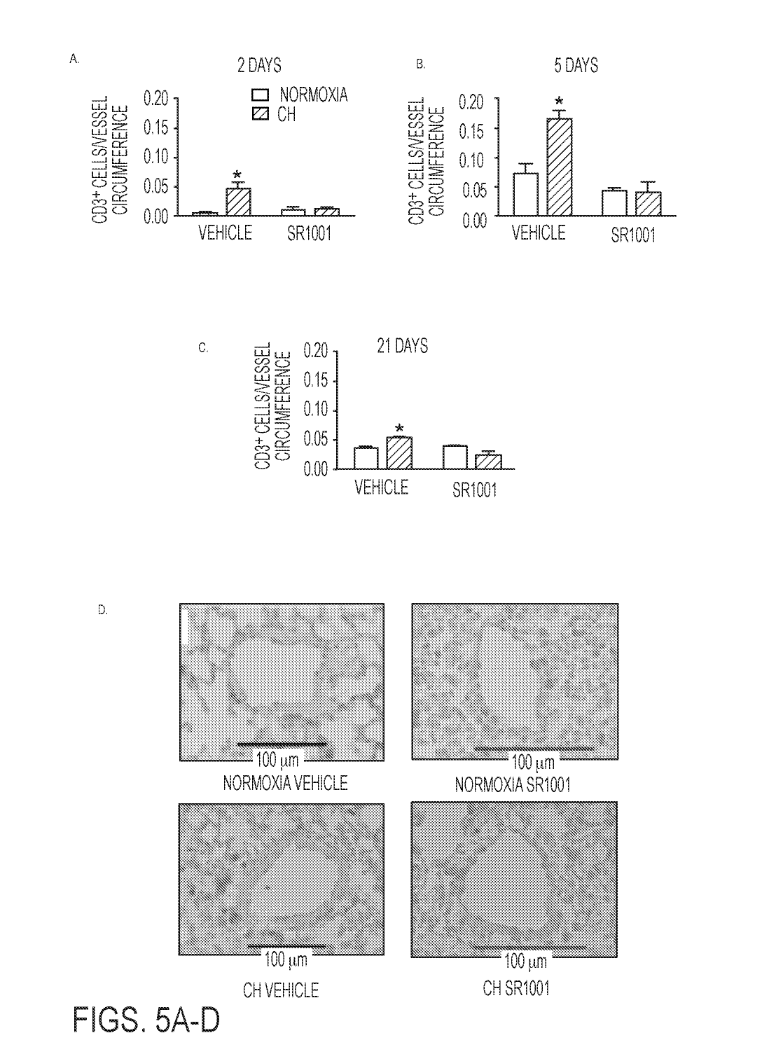

T_{H}17 cell development depends on signaling from the nuclear receptors RORα and RORγt (Ivanov et al., 2006; Yang et al., 2008). The selective inverse agonist SR1001 inhibits the activity of these nuclear receptors (Solt et al., 2012; Solt et al., 2011) and inhibits T_{H}17 cell development both in vitro and in vivo (Solt et al., 2011). To determine the contribution of T_{H}17 cells to CH-induced lung inflammation, WT mice were treated with SR1001 and exposed to CH for 2, 5 or 21 days. T cells were examined in lung sections by immunohistochemistry detection of the pan-T cell marker CD3. Following conditions of CH, CD3⁺ T cells were found in the pulmonary arterial perivascular region of vehicle-treated mice (FIG. 5A-D). On the contrary, mice treated with SR1001 and exposed to CH show a significant reduction in perivascular T cells at all three time points (FIG. 5A-D), suggesting that a significant number of the perivascular T cells may be cells reliant upon RORγτ signaling, such as T_{H}17 cells. The increase in perivascular T cells was not due to overall increase in parenchymal T cells or total lung T cells.

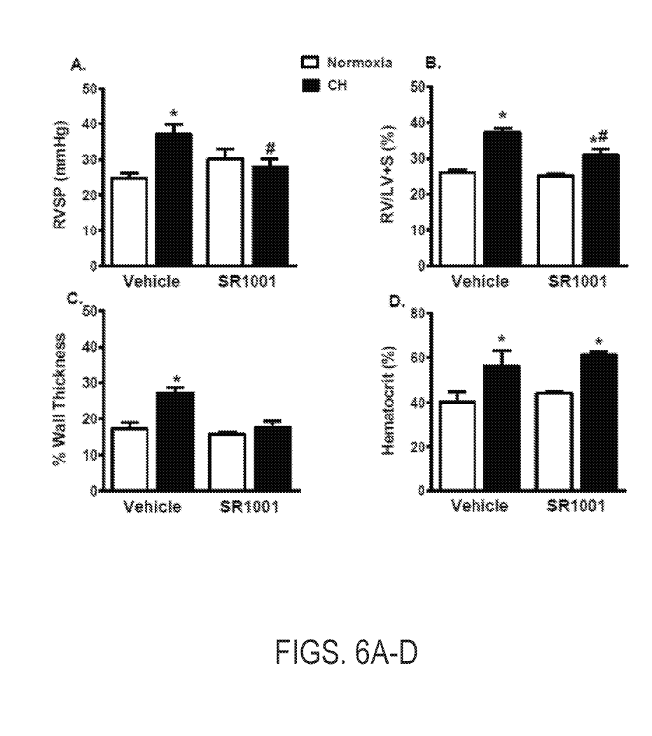

More importantly, inhibition of T_{H}17 cell development, by SR1001 administration, attenuated CH-induced increases in RVSP, RV and pulmonary arterial remodeling without affecting the polycythemic response (FIG. 6). These results support our hypothesis that TH17 cells contribute to CH-induced PH.

T_{H}17 cell inhibition attenuates increases in RVSP in established PH.

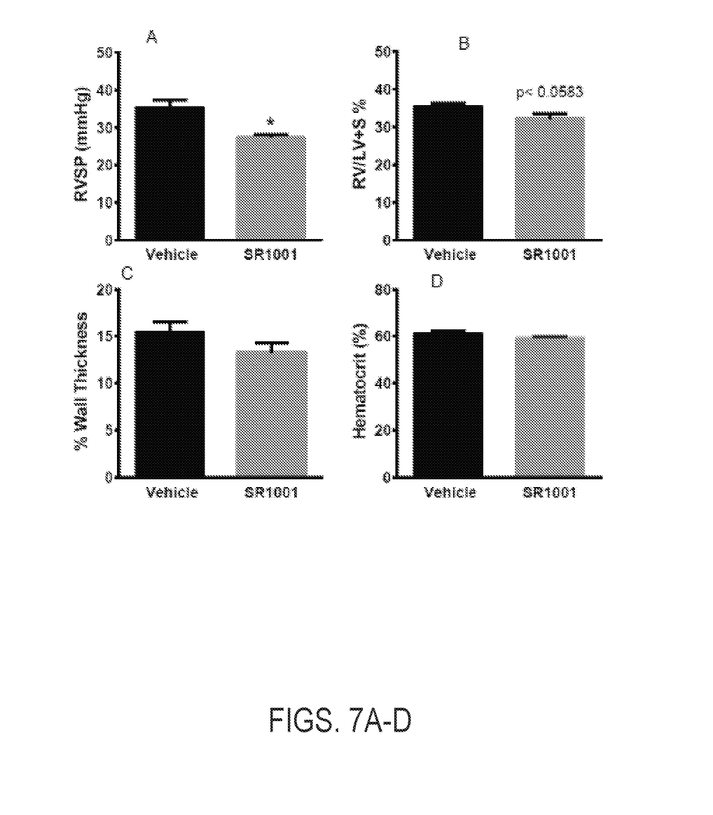

To follow up on the immunologic timing of events, studies were undertaken to understand whether PH due to CH might be reversed once already established by inhibiting T_{H}17 cell development. Therefore, after 3 weeks of CH, osmotic pumps containing SR1001 (0.625 mg/day for 25 g mouse). were implanted sub-cutaneously and animals were immediately returned to CH for an additional 2 weeks (5 weeks of CH total). Inhibition of T_{H}17 cell development with SR1001 significantly decreased already elevated RVSP (FIG. 7A). A strong trend for a decrease in RV remodeling (p<0.0583) was observed but no effect on pulmonary arterial remodeling was detected (FIGS. 7B and C). There was also a significant decrease in perivascular T cells in the lungs of mice receiving SR1001 (FIG. 7D). These results support a crucial role for T_{H}17 cells in PH pathogenesis

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,277,175;
U.S. Pat. No. 5,284,133;
U.S. Pat. No. 5,299,566;
U.S. Pat. No. 5,355,872;
U.S. Pat. No. 5,660,166;
U.S. Pat. No. 5,797,389;
U.S. Pat. No. 6,016,974;
U.S. Pat. No. 6,025,395,
U.S. Pat. No. 6,041,776;
U.S. Pat. No. 6,044,841;
U.S. Pat. No. 6,241,159;
U.S. Pat. No. 6,354,516;
U.S. Pat. No. 6,357,671;
U.S. Pat. No. 6,921,020;
U.S. Pat. No. 6,926,208;
U.S. Pat. No. 6,968,840;
U.S. Pat. No. 6,978,941;
U.S. Pat. No. 7,040,549;
U.S. Pat. No. 7,083,112;
U.S. Pat. No. 7,104,463;
U.S. Pat. No. 7,360,536
U.S. Published Patent Application Nos. 20020020412; 20020020409; and 20090134235 WO 2011/115892
Barst et al., A comparison of continuous intravenous epoprostenol (prostacyclin) with conventional therapy for primary pulmonary hypertension. N. Engl. J Med., 334:296-301, 1996.
Beurel et al., Inflammatory T Helper 17 Cells Promote Depression-like Behavior in Mice. Biological Psychiatry 2013; 73(7):622-30.
Bierer et al., NFATc3 Is Required for Chronic Hypoxia-induced Pulmonary Hypertension in Adult and Neonatal Mice. American Journal of Physiology—Lung Cellular and Molecular Physiology 2011.
Bolignano et al., Pulmonary Hypertension in CKD, Am. J. Kidney Dis., 61:612-622, 2013.
Cheng X, Yu X, Ding Yj, Fu Qq, Xie Jj, Tang Tt, Yao R, Chen Y and Liao Yh. The Th17/Treg imbalance in patients with acute coronary syndrome. Clinical Immunology 127: 89-97, 2008.
Chen et al., Neutralization of interleukin-17A delays progression of silica-induced lung inflammation and fibrosis in C57BL/6 mice. Toxicology and Applied Pharmacology 2014; 275(1):62-72.
Cuttica et al., Perivascular T-Cell Infiltration Leads to Sustained Pulmonary Artery Remodeling after Endothelial Cell Damage. American Journal of Respiratory Cell and Molecular Biology 2011; 45(1):62-71.
Daley et al., Pulmonary arterial remodeling induced by a Th2 immune response. J Exp Med 2008; 205(2):361-72.
De Frutos et al., NFATc3 mediates chronic hypoxia-induced pulmonary arterial remodeling with alpha-actin up-regulation. J Biol Chem 2007; 282:15081-9.
De Frutos et al., NFATc3 is required for intermittent hypoxia-induced hypertension. Am J Physiol Heart Circ Physiol 2008; 294(5):H2382-H2390.
Deng et al., Familial primary pulmonary hypertension (gene PPH1) is caused by mutations in the bone morphogenetic protein receptor-II gene. Am. J. Hum. Genet., 67:737-44, 2000.
Dang E V, Barbi J, Yang H Y, Jinasena D, Yu H, Zheng Y, Bordman Z, Fu J, Kim Y, Yen H R, Luo W, Zeller K, Shimoda L, Topalian S L, Semenza G L, Dang C V, Pardoll D M and Pan F. Control of TH17/Treg Balance by Hypoxia-Inducible Factor 1. Cell 146: 772-784, 2011.
Eid R E, Rao D A, Zhou J, Lo Sf, Ranjbaran H, Gallo A, Sokol S I, Pfau S, Pober J S and Tellides G. Interleukin-17 and Interferon-gamma Are Produced Concomitantly by Human Coronary Artery-coInfiltrating T Cells and Act Synergistically on Vascular Smooth Muscle Cells. Circulation 119: 1424-1432, 2009.
Eisen S A, Wedner H J and Parker C W. Isolation of pure human peripheral blood T-lymphocytes using nylon wool columns. Immunol Commun 1: 571-577, 1972.
Franco et al., Magnetic Resonance Imaging and Invasive Evaluation of Development of Heart Failure in Transgenic Mice With Myocardial Expression of Tumor Necrosis Factor-+¦. Circulation 1999; 99(3):448-54.
Fults et al., J Pharm. Pharmacol., 43:726-8, 1991
Galie et al., 2002
Galie et al., Ambrisentan therapy for pulmonary arterial hypertension. J Am. Coll. Cardiol., 46:529-535, 2005.
Handbook of Pharmaceutical Salts: Properties, and Use, (P. H. Stahl & C. G. Wermuth eds., Verlag Helvetica Chimica Acta, 2002).
Hashimoto-Kataoka T, Hosen N, Sonobe T, Arita Y, Yasui T, Masaki T, Minami M, Inagaki T, Miyagawa S, Sawa Y, Murakami M, Kumanogoh A, Yamauchi-Takihara K, Okumura M, Kishimoto T, Komuro I, Shirai M, Sakata Y and Nakaoka Y. Interleukin-6/interleukin-21 signaling axis is critical in the pathogenesis of pulmonary arterial hypertension. Proc Natl Acad Sci USA 112: E2677-E2686, 2015.
Hautefort A, Girerd B, Montani D, Cohen K S, Price L, Lambrecht B N, Humbert M and Perros F. Th17 polarization in pulmonary arterial hypertension. Chest 2014.
Hiromatsu K, Yoshikai Y, Matsuzaki G, Ohga S, Muramori K, Matsumoto K, Bluestone J A, Nomoto K. A protective role of gamma/delta T cells in primary infection with Listeria monocytogenes in mice. J Exp Med 1992; 175(1):49-56.
Hopkins N, McLoughlin P. The structural basis of pulmonary hypertension in chronic lung disease: remodelling, rarefaction or angiogenesis? Journal of Anatomy 2002; 201(4):335-48.
Ivanov I I, McKenzie B S, Zhou L, Tadokoro C E, Lepelley A, Lafaille J J, Cua D J and Littman D R. The Orphan Nuclear Receptor RORγτ Directs the Differentiation Program of Proinflammatory IL-17+T Helper Cells. Cell 126: 1121-1133, 2006.
Kamiyama et al., Coexpression of CCR6 and CD146 (MCAM) is a marker of effector memory T-helper 17 cells. The Journal of Dermatology 2012; 39(10):838-42.
Kataoka et al., Oral sildenafil improves primary pulmonary hypertension refractory to epoprostenol. Circ. J., 69:461-465, 2005.
Lahl et al., Selective depletion of Foxp3+ regulatory T cells induces a scurfy-like disease. J Exp Med 2007; 204(1):57-63.
Langleben et al., STRIDE 1: Effects of the Selective ETA Receptor Antagonist, Sitaxsentan Sodium, in a Patient Population with Pulmonary Arterial Hypertension that meets Traditional Inclusion Criteria of Previous Pulmonary Arterial Hypertension Trials. J. Cardiovasc. Pharmacol., 44:S80-S84.9, 2004.
Lee et al., Sildenafil for pulmonary hypertension. Ann. Pharmacother., 39:869-884, 2005.
Lindén A. Interleukin-17 and airway remodelling. Pulmonary Pharmacology & Therapeutics 19: 47-50, 2006.
Madhur M S, Lob H E, McCann L A, Iwakura Y, Blinder Y, Guzik T J and Harrison D G. Interleukin 17 Promotes Angiotensin II-Induced Hypertension and Vascular Dysfunction. Hypertension 55: 500-507, 2010.
March's Advanced Organic Chemistry: Reactions, Mechanisms, and Structure, 2007.
Mazzolai et al., Endogenous Angiotensin II Induces Atherosclerotic Plaque Vulnerability and Elicits a Th1 Response in ApoE−/− Mice. Hypertension 2004; 44(3):277-82.
McGoon et al., Screening, early detection, and diagnosis of pulmonary arterial hypertension: ACCP evidence-based clinical practice guidelines. Chest, 126:14S-34S, 2004.
McLaughlin et al., Reduction in pulmonary vascular resistance with long-term epoprostenol (prostacyclin) therapy in primary pulmonary hypertension. N. Engl. J. Med., 338:273-277, 1998.
McLaughlin and Rich, Pulmonary hypertension. Curr. Probl. Cardiol., 29:575-634, 2004.
McLean et al., Anal Chem., 72:4796-804, 2000.
Mombaerts et al., RAG-1-deficient mice have no mature B and T lymphocytes. Cell 1992; 68(5):869-77.
Oudiz et al., Treprostinil, a prostacyclin analogue, in pulmonary arterial hypertension associated with connective tissue disease. Chest, 126:420-427, 2004.
Park et al., Interleukin 13- and interleukin 17A-induced pulmonary hypertension phenotype due to inhalation of antigen and fine particles from air pollution. Pulmonary Circulation 2014; 4(4):654-68.
Poor H D, Girgis R, Studer S M. World Health Organization Group III pulmonary hypertension. Progress in Cardiovascular Diseases 2012; 55(2): 119-27.
Repka et al., Hot-Melt Extrusion Technology, Encyclopedia of Pharmaceutical Technology. 2nd Ed. New York: Marcel Dekker, Inc.; 2002.
Rubin et al., 2002
Savale L, Tu L, Rideau D, Izziki M, Maitre B, Adnot S and Eddahibi S. Impact of interleukin-6 on hypoxia-induced pulmonary hypertension and lung inflammation in mice. Respiratory Research 10: 6, 2009.
Shao et al., Imbalance of T-Cell Subsets in Angiotensin II-Infused Hypertensive Rats With Kidney Injury. Hypertension 2003; 42(1):31-8.
Smith et al., IL-17A Inhibits the Expansion of IL-17A-Producing T Cells in Mice through “Short-Loop” Inhibition via IL-17 Receptor. The Journal of Immunology 2008; 181(2):1357-64.
Solt et al., Suppression of TH17 differentiation and autoimmunity by a synthetic ROR ligand. Nature 2011; 472(7344):491-4.
Solt L A, Kumar N, He Y, Kamenecka T M, Griffin P R and Burris T P. Identification of a Selective RORγτ Ligand That Suppresses T_{H}17 Cells and Stimulates T Regulatory Cells. ACS Chem Biol 7: 1515-1519, 2012.
Solt L A, Kumar N, Nuhant P, Wang Y, Lauer J L, Liu J, Istrate M A, Kamenecka T M, Roush W R, Vidovic D, Schurer S C, Xu J, Wagoner G, Drew P D, Griffin P R and Burns T P. Suppression of TH17 differentiation and autoimmunity by a synthetic ROR ligand. Nature 472: 491-494, 2011.
Strejan et al., J Neuroimmunol. 7(27), 1984.
Sutton et al., IL-17-producing gdT cells and innate lymphoid cells. Eur J Immunol 2012; 42(9):2221-31.
Tesmer L A, Lundy S K, Sarkar S and Fox D A. Th17 cells in human disease. Immunol Rev 223: 87-113, 2008.
Urboniene et al., Distribution of gd and other T-lymphocyte subsets in patients with chronic obstructive pulmonary disease and asthma. Respiratory Medicine 2013; 107(3):413-23.
Vachiery and Naeije, Treprostinil for pulmonary hypertension. Expert Rev. Cardiovasc. Ther., 2:183-191, 2004.
Vargas-Rojas MaIs, Ram+ [symbol] rez-Venegas A, Lim+¦n-Camacho L, Ochoa L, Hern+índez-Zenteno R and Sansores RlH. Increase of Th17 cells in peripheral blood of patients with chronic obstructive pulmonary disease. Respiratory Medicine 105: 1648-1654, 2011.
van der Heyde et al., Gamma delta T cells function in cell-mediated immunity to acute blood-stage Plasmodium chabaudi adami malaria. The Journal of Immunology 1995; 154(8):3985-90.
Vecellio et al., J Aerosol. Med., 14:107-14, 2001.
Veldhoen M, Hirota K, Christensen J, O'Garra A, Stockinger B. Natural agonists for aryl hydrocarbon receptor in culture medium are essential for optimal differentiation of Th17 T cells. J Exp Med 2009; 206(1):43-9.
Waldrep and Dhand, 2008.
Yang X O, Pappu B, Nurieva R, Akimzhanov A, Kang H S, Chung Y, Ma L, Shah B, Panopoulos A D, Schluns K, Watowich S S, Tian Q, Jetten A M and Dong C. TH17 lineage differentiation is programmed by orphan nuclear receptors RORα and RORγ. Immunity 2428: 29-39, 2008.

1. A method for treating or preventing pulmonary hypertension in a subject comprising administering an effective amount of a retinoic acid receptor-related orphan nuclear receptor (ROR) inhibitor.

2. The method of claim 1, wherein the ROR inhibitor is a RORα or RORγτ inhibitor.

3. The method of claim 1, wherein the ROR inhibitor is a selective RORγτ inhibitor.

4. The method of claim 1, wherein the effective amount of the ROR inhibitor administered to the subject is an amount effective to reduce T_{H}17 cell levels, reduce T_{H}17 cell activity or reduce resting mean pulmonary artery pressure in the subject.

5. The method of claim 1, wherein the subject has pulmonary hypertension.

6. The method of claim 1, wherein the subject has or has been previous diagnosed with chronic obstructive pulmonary disease (COPD), acute respiratory distress syndrome (ARDS), acute lung injury (ALI) or chronic lower respiratory diseases (CLRD).

7. (canceled)

8. (canceled)

9. The method of claim 1, further comprising administering at least a second therapeutic to the subject.

10. The method of claim 9, wherein the second therapeutic is a vasodilator, a prostanoid, an endothelin receptor antagonist, a phosphodiesterase-5 inhibitors or a sGC stimulator.

11. The method of claim 9, wherein the second therapeutic comprises bosentan, macitentan, prostacyclin, sildenafil, tadalafil, treprostinil, iloprost or riociguat.

12. (canceled)

13. (canceled)

14. The method of claim 1, wherein the ROR inhibitor comprises a compound having the formula (I): 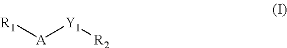
wherein:
A is —CH₂—, —S(O)₂NR₃— or —C(O)NR₃—;
R₃ is hydrogen, alkyl_{(C≤12)}, substituted alkyl_{(C≤12)}, acyl_{(C≤12)}, or substituted acyl_{(C≤12)};
Y₁ is arenediyl_{(C≤18)}, heteroarenediyl_{(C≤18)}, or a substituted version thereof;
R₁ is aryl_{(C≤12)}, heteroaryl_{(C≤12)}, heterocycloalkyl_{(C≤12)}, or a substituted version of either of these groups; or an aryl_{(C≤12)}, heteroaryl_{(C≤12)}, heterocycloalkyl_{(C≤12)}, or a substituted version of either of these groups wherein the group is further substituted with an acyl_{(C≤8)}, amido_{(C≤8)}, alkylsulfonyl_{(C≤8)}, arylsulfonyl_{(C≤8)}, heteroarylsulfonyl_{(C≤8)}, alkylsulfonylamino_{(C≤8)}, arylsulfonylamino_{(C≤8)}, heteroarylsulfonylamino_{(C≤8)}, or a substituted version of any of these groups;
R₂ is 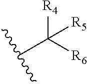
wherein:
R₄ and R₆ is a haloalkyl_{(C≤6)}; and
R₅ is hydroxy, alkoxy_{(C≤6)}, substituted alkoxy_{(C≤6)}, acyloxy_{(C≤6)}, or substituted acyloxy_{(C≤6)};
or a pharmaceutically acceptable salt thereof.

15. The method of claim 14, wherein R₁ is heteroaryl_{(C≤12)} or a substituted heteroaryl_{(C≤12)}.

16. The method of claim 15, wherein R₁ is:

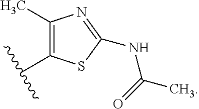 [SMILES: *c1sc(NC(C)=O)nc1C]

17. The method of claim 14, wherein R₁ is heterocycloalkyl_{(C≤12)} or substituted heterocycloalkyl_{(C≤12)}.

18. The method of claim 17, wherein R₁ is:

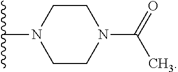 [SMILES: *N1CCN(C(C)=O)CC1]

19. The method according to claim 14, wherein A is —S(O)₂NR₃—.

20. The method according to claim 14, wherein A is —CH₂—.

21. The method according to claim 14, wherein R₃ is hydrogen.

22. The method according to claim 14, wherein Y₁ is arenediyl_{(C≤18)}.

23. The method of claim 22, wherein Y₁ is:

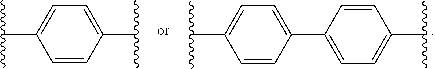 [SMILES: *c1ccc(*)cc1 ; *c1ccc(-c2ccc(*)cc2)cc1]

24. The method according to claim 14, wherein R₄ is —CF₃.

25. The method according to claim 14, wherein R₆ is —CF₃.

26. The method according to claim 14, wherein R₅ is hydroxy.

27. The method according to claim 14, wherein R₂ is:

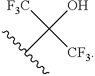 [SMILES: *C(O)(C(F)(F)F)C(F)(F)F]

28. The method of claim 14, wherein Y₁ and R₂ are taken together and are:

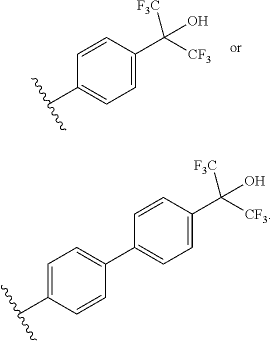 [SMILES: *c1ccc(C(O)(C(F)(F)F)C(F)(F)F)cc1 ; *c1ccc(-c2ccc(C(O)(C(F)(F)F)C(F)(F)F)cc2)cc1]

29. The method according to claim 14, wherein the compound is further defined as: 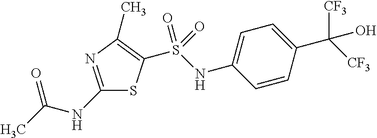 [SMILES: CC(=O)Nc1nc(C)c(S(=O)(=O)Nc2ccc(C(O)(C(F)(F)F)C(F)(F)F)cc2)s1]
or a pharmaceutically acceptable salt thereof.

30. The method according to claim 14,
wherein the compound is further defined as: 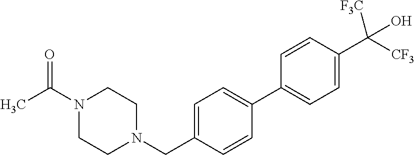 [SMILES: CC(=O)N1CCN(Cc2ccc(-c3ccc(C(O)(C(F)(F)F)C(F)(F)F)cc3)cc2)CC1]
or a pharmaceutically acceptable salt thereof.

31. A method for treating or preventing pulmonary hypertension in a subject comprising administering an effective amount of a compound of formula I to the subject, wherein the compound has the formula: 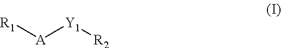
wherein:
A is —CH₂—, —S(O)₂NR₃— or —C(O)NR₃—;
R₃ is hydrogen, alkyl_{(C≤12)}, substituted alkyl_{(C≤12)}, acyl_{(C≤12)}, or substituted acyl_{(C≤12)};
Y₁ is arenediyl_{(C≤18)}, heteroarenediyl_{(C≤18)}, or a substituted version thereof;
R₁ is aryl_{(C≤12)}, heteroaryl_{(C≤12)}, heterocycloalkyl_{(C≤12)}, or a substituted version of either of these groups; or an aryl_{(C≤12)}, heteroaryl_{(C≤12)}, heterocycloalkyl_{(C≤12)}, or a substituted version of either of these groups wherein the group is further substituted with an acyl_{(C≤8)}, amido_{(C≤8)}, alkylsulfonyl_{(C≤8)}, arylsulfonyl_{(C≤8)}, heteroarylsulfonyl_{(C≤8)}, alkylsulfonylamino_{(C≤8)}, arylsulfonylamino_{(C≤8)}, heteroarylsulfonylamino_{(C≤8)}, or a substituted version of any of these groups;
R₂ is 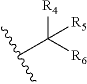
wherein:
R₄ and R₆ is a haloalkyl_{(C≤6)}; and
R₅ is hydroxy, alkoxy_{(C≤6)}, substituted alkoxy_{(C≤6)}acyloxy_{(C≤6)}, or substituted acyloxy_{(C≤6)};
or a pharmaceutically acceptable salt thereof.

32.-52. (canceled)

53. A composition comprising a nebulized solution of a compound of formula I in a pharmaceutically acceptable carrier, wherein the compound has the formula: 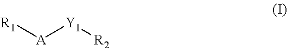
wherein:
A is —CH₂—, —S(O)₂NR₃— or —C(O)NR₃—;
R₃ is hydrogen, alkyl_{(C≤12)}, substituted alkyl_{(C≤12)}, acyl_{(C≤12)}, or substituted acyl_{(C≤12)};
Y₁ is arenediyl_{(C≤18)}, heteroarenediyl_{(C≤18)}, or a substituted version thereof;
R₁ is aryl_{(C≤12)}, heteroaryl_{(C≤12)}, heterocycloalkyl_{(C≤12)}, or a substituted version of either of these groups; or an aryl_{(C≤12)}, heteroaryl_{(C≤12)}, heterocycloalkyl_{(C≤12)}, or a substituted version of either of these groups wherein the group is further substituted with an acyl_{(C≤8)}, amido_{(C≤8)}, alkylsulfonyl_{(C≤8)}, arylsulfonyl_{(C≤8)}, heteroarylsulfonyl_{(C≤8)}, alkylsulfonylamino_{(C≤8)}, arylsulfonylamino_{(C≤8)}, heteroarylsulfonylamino_{(C≤8)}, or a substituted version of any of these groups;
R₂ is 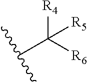
wherein:
R₄ and R₆ is a haloalkyl_{(C≤6)}; and
R₅ is hydroxy, alkoxy_{(C≤6)}, substituted alkoxy_{(C≤6)}acyloxy_{(C≤6)}, or substituted acyloxy_{(C≤6)};
or a pharmaceutically acceptable salt thereof.

54.-86. (canceled)

87. A method of treating or preventing PH comprising administering an effective amount of a composition according to any one claim 53 to the airway of a subject in need of treatment.
